(19) United States
(12) Patent Application Publication
(10) Pub. No.: US 2012/0202284 A1
(43) Pub. Date: Aug. 9, 2012

(54) CELL SORTING APPARATUS AND METHOD

(75) Inventors: John Gal, New South Wales (AU); Robert Kery, New South Wales (AU); Sung-Wei Chen, Singapore (SG)

(73) Assignee: Empire Technology Development LLC

(21) Appl. No.: 13/203,933

(22) PCT Filed: Feb. 7, 2011
(86) PCT No.: PCT/AU11/00123

§ 371 Date: Aug. 30, 2011

(51) Int. Cl. C12M 1/00; C12N 5/071

(52) U.S. Cl. 435/325; 435/283.1

(57) ABSTRACT

Apparatus and method for sorting cells is provided in which two or more cells are delivered along a flow path, the two or more cells including a first type of cells having a first mechanical stiffness and at least a second type of cells having a second mechanical stiffness different from the first mechanical stiffness. A reflection surface is provided across the flow path at an oblique angle relative to the flow path and configured to reflect the cells at different reflection angles relative to the flow path dependent on the mechanical stiffness of the cells.

### BACKGROUND

Existing methods of cell identification and separation are problematic and often unsatisfactory. For example, difficulties experienced in detection and sorting of stem cells are a significant barrier to the use of stem cells for the manufacturing of replacement organs.

Current sorting techniques such as fluorescence-activated cell sorting (FACS) or magnetic-activated cell sorting (MACS) require that cells are modified by the attachment of a suitable marker for subsequent detection.

Such techniques can suffer from slow sorting speeds, low cell yields and cell damage.

### SUMMARY

By way of non-limiting examples, embodiments are now disclosed. In a first embodiment, apparatus for sorting cells is provided, including a cell delivery device, a reflection surface, a first collection area and a second collection area. In this first embodiment, the delivery device is configured to deliver two or more cells along a flow path, the two or more cells including a first type of cells having a first mechanical stiffness and at least a second type of cells having a second mechanical stiffness different from the first mechanical stiffness. The reflection surface is provided across the flow path at an oblique angle relative to the flow path and configured to reflect the cells at reflection angles relative to the flow path. In this first embodiment, the first collection area is positioned to receive the first type of cells reflected from the reflection surface substantially at a first reflection angle; and the second collection area is positioned to receive the second type of cells reflected from the reflection surface substantially at a second reflection angle different from the first reflection angle. The difference in the first and second reflection angles is dependent on the mechanical stiffness of the first and second types of cells.

In a second embodiment, a method of sorting cells is disclosed. The method includes causing two or more cells travelling along a flow path to contact a reflection surface and to rebound at different reflection angles, the cells including a first type of cells having a first mechanical stiffness and at least a second type of cells having a second mechanical stiffness different from the first mechanical stiffness; collecting the first type of cells reflected from the reflection surface substantially at a first reflection angle; and collecting the second type of cells reflected from the reflection surface substantially at a second reflection angle different from the first reflection angle. The first and second reflection angles are different dependent on the mechanical stiffness properties of the first and second types of cells.

In a third embodiment, another apparatus for sorting cells is disclosed. The apparatus in the third embodiment includes means for producing a flow of two or more cells, impact means, upon which the cells are impactable, wherein impact with said impact means causes the cells to travel to different locations relative to the impact means as a result of differences in kinetic energy loss of the cells upon impact; and means for collecting the cells at the different positions.

### BRIEF DESCRIPTION OF THE FIGURES

FIG. 1 illustrates an example apparatus for sorting cells;

FIG. 2 illustrates another example apparatus for sorting cells;

FIG. 3 illustrates a further example apparatus for sorting cells;

FIG. 4 illustrates yet another example apparatus for sorting cells; and

FIG. 5 illustrates velocity components of a cell reflected at a surface.

### DETAILED DESCRIPTION

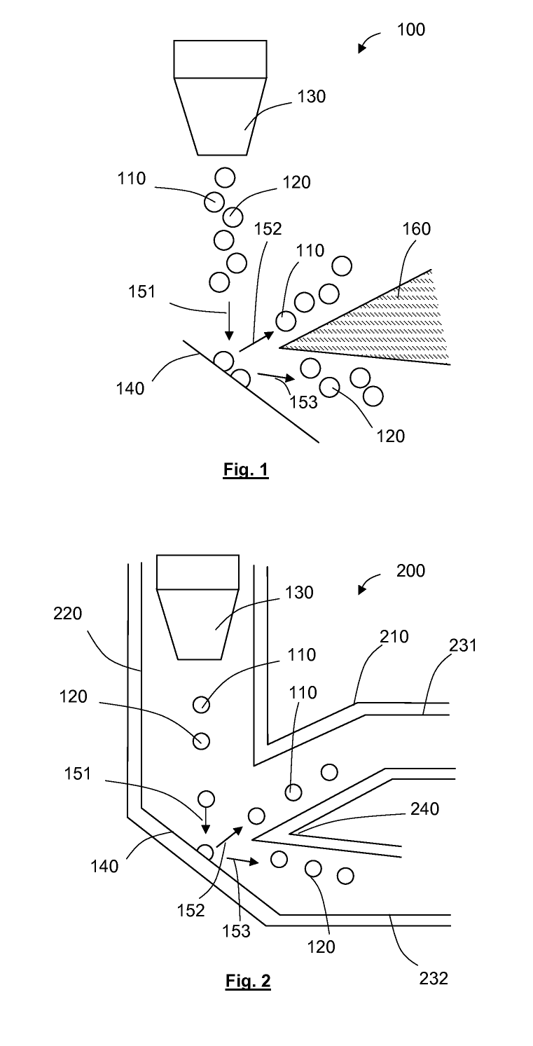

FIG. 1 illustrates an example apparatus 100 for sorting at least two cells 110, 120. The apparatus includes a nozzle 130 for delivering the cells 110, 120 towards a reflection surface 140, the reflection surface 140 being disposed at an oblique angle relative to the flow path of the delivered cells as they collide with the reflection surface. The flow path of the cells incident on the surface 140 (i.e., the incident flow path) is indicated generally by a first arrow 151 in FIG. 1. The cells may be delivered from the nozzle 130 at a rate of up to 40,000 cells/second, although other cell rates may be used. The cells may be enclosed in fluid droplets as they are delivered from the nozzle. The nozzle 130 may be a pipe, funnel or spout, or other device, having an opening from which cells can be delivered along a flow path. The reflection surface 140 may be part of any object against which the cells can impact in a manner resulting in reflection (rebounding) of the cells from that surface, reflection or rebounding meaning that the cells bound or spring from the surface after impact with the surface at least partially or completely as a result of the force of the impact. The object having the reflection surface may be a table, board, panel, block, shelf, projection or take other forms.

In this example, the cells 110, 120 include at least two types of cells, a first type of cells 110 having substantially a first mechanical stiffness and at least a second type of cells 120 having substantially a second mechanical stiffness different from the first mechanical stiffness.

The apparatus 100 is configured to sort at least the first type of cells 110 from the second type of cells 120 partially or completely in view of the different mechanical stiffness of the different cell types. Sorting may include the separation or partial separation of the at least first and second types of cells, the cells being separated, as a result of the sorting, into two or more different cell samples. The separation may be partial in the sense that, even after sorting, there may be cells of both the first and second types of cells in one or more of the cell samples. In this example, the second mechanical stiffness is lower than the first mechanical stiffness. By delivering the cells 110, 120 towards the surface 140, the cells can impact (i.e., collide) with the surface 140 and reflect from the surface 140 at an angle relative to the incident flow path 151. At least due to the difference in mechanical stiffness, the second type of cells 120 in this example reflect at a greater reflection angle relative to the normal of the reflection surface 140 than the first type of cells 110. Thus, at least the first and second types of cells 110, 120 can travel along diverging reflection flow paths, after reflecting from the surface 140, the reflection flow paths indicated generally by second and third arrows 152, 153 in FIG. 1. (Features of the apparatus, cells and flow paths are illustrated in the accompanying Figures in a manner to aid understanding of the examples and the features are not necessarily depicted at a scale or relative orientation that would be used in practice).

It is understood that there may be variations in the stiffness of cells within a population of a single type of cells. In general, the differences in stiffness between the different types of cells to be sorted will be greater than the differences in stiffness in a pure population of at least one of the different types of cells. As an example, with reference to Pajerowski, J. D., et al., Physical plasticity of the nucleus in stem cell differentiation, PNAS (2007) Oct. 2, vol. 104, no. 40, 15619-15624, FIG. 1, variation in stiffness of differentiated and undifferentiated stem cells, in respective populations of these types of cells, may be about +/−50% of the average stiffness in the populations. This variation may be partly due to the variation in properties of cells and partly due to measurement error. Thus, for undifferentiated and differentiated stem cell populations, each population exhibiting a variation in average stiffness, efficient sorting may be achieved where the difference between the average stiffness of the two different populations is at least about 100%. With reference to Tan S. C. et al., Viscoelastic behaviour of human mesenchymal stem cells, BMC Cell Biol. 2008 Jul. 22; 9:40, and Pajerowski, J. D., et al, the elastic modulus of stem cells may range from approximately 900 Pa for undifferentiated cells to 5400 Pa for fully differentiated cells. Since this gives an approximately 600% (6-fold) difference in stiffness between these two types of cells, any +/−50% variation of the average stiffness in respective populations of each of these two types of cells may not be significant. Thus, efficient sorting of undifferentiated and differentiated stem cells is described as one example of types of cells that may be sorted in apparatus and methods described herein.

The reflection surface may be a rigid surface or a non-rigid surface. When a rigid surface is used there may be substantially no flexing or movement of the surface upon impact with the cells. The surface may be sufficiently rigid to minimise absorption of energy from the cells. Any flexibility may result in loss of rebound velocity and therefore diminish the yield. Examples of materials that may have sufficient rigidity include thermoplastics and stainless steel. The reflection surface may be entirely coated, partially coated or uncoated. For example, a coating may be applied at least partially to the reflection surface to alter friction properties of the surface. For example, the coating may be provided to increase the coefficient of friction of the surface, to reduce or prevent slippage of the cells upon impact with the surface, or to decrease the coefficient of friction, to reduce or prevent sticking of the cells upon impact with the surface. The coefficient of friction of the reflection surface may additionally or alternatively be altered by providing a surface with a particular roughness, with or without a coating applied to the surface. By reducing or preventing sliding or sticking of the cells, the possibility of cells reflecting in an unpredictable manner may be reduced or prevented, making it more straightforward to sort the cells. As another example, the reflection surface may additionally or alternatively be coated with a hydrophobic and/or hydrophilic coating to reduce or prevent sticking or slipping of the cells. On the whole, the surface may be configured, whether coated or not, to minimise slippage and/or sticking of cells and to provide consistent uniformity and rigidity properties for different types of cells that are to impact with the surface. Coatings that may be suitable include but are not limited to Teflon™ (polytetrafluoroethylene (PTFE)) or other fluorinated polymers, which, in addition to their friction properties, may have desired hydrophobicity properties. Polymers such as but not limited to 2-hydroxyethyl methacrylate (HEMA) and other methacrylates may be used to increase hydrophilicity Roughened surfaces may include physically post-treated polymer surfaces, such as physically abraided polyethylene, or a patterned surface created by micromachining ion milled film, for example.

Prior to delivery from the nozzle, the cells may be combined with a column of pressurized sheath fluid. A piezo transducer may be used to break up the suspension of cells in the fluid into a rapidly moving stream of droplets, ejected from the nozzle. The nozzle may be a conventional nozzle used in FACS equipment, or otherwise. In an illustrative embodiment, a FACS nozzle arrangement which may be used in the present apparatus is described in Derek Davies, Chapter 5: Cell Sorting by Flow Cytometry in Flow Cytometry: Principles and Applications, edited by M. G. Macey, Humana Press Inc., Totwa N.J. The flow rate of cells will depend on the sheath pressure, the nozzle size, and the number of cells. However, as an example, with a nozzle size of 50 microns and a sheath pressure of 80 psi, 160,000 drops per second may be delivered from the nozzle. If there is, for example, 1 cell in every four drops, 40,000 cells per second may therefore be delivered from the nozzle, for example. The velocity of the cells will also vary depending on the sheath pressure, but may be a velocity of approximately 10 m/s for a sheath pressure of 12 psi and approximately 50m/s for sheath pressure of 80 psi. In general, however, many different approaches may be taken to delivering a flow of cells to the reflection surface.

One or more separation elements, particularly a wedge 160 in this example, can be employed to maintain separation of cells 110, 120 as they travel at different reflection angles along the different reflection flow paths 152, 153 after reflecting from the reflection surface 140. The one or more separation elements may define two or more collecting areas for the reflected cells. The wedge 160 in this example has a wedge angle corresponding substantially to the difference in angle of the reflection angles of the first and second cell types 110, 120, although other wedge angles may be employed, e.g., to adjust a degree of divergence between the flow paths 152, 153. To change the wedge angle, the wedge 160 may be interchangeable with other wedges or the wedge itself may be adjustable or moveable. The wedge, or another type of separation element, may be adjusted or moved in accordance with the angle of difference between the reflection angles of the first and second cell types (e.g., so that the wedge angle substantially matches the angle of difference), or to adjust the degree of divergence between the flow paths of the reflected cells as desired. As one example, the wedge may be adjustable by comprising two opposing walls, angled relative to each other, the walls converging to an apex of the wedge shape, wherein the walls are pivotably connected at the apex, optionally by a hinge. A mechanism, such as a screw mechanism or linear actuator, for example, located between the two opposing walls, may be operable to relatively pivot the walls, although other mechanisms may be employed.

One or more separation elements other than a wedge may be used in the example illustrated in FIG. 1, or in other examples. In general, a separation element may be any device or mechanism by which cells can be kept apart from each other after they are reflected from the one or more reflection surfaces. The cells may be kept apart by the one or more separation elements while they are moving and/or while they are stationary following reflection from the one or more reflection surfaces. In addition to keeping the separated cells apart, the one or more separation elements may serve to channel or move the cells in a desired direction after they reflect from the reflection surface, e.g. towards collection containers or otherwise. A separation element may be provided by a wedge, shelf, ledge, wall, barrier, rod or other element capable of maintaining cells apart.

The orientation and/or distance of the separation element from the reflection surface may be chosen depending on one or more factors such as but not limited to the incident angle of the cells on the reflection surface, the width of the stream of cells incident on the reflection surface (the ‘beam width’), the difference in the reflection angles between the different cell types reflecting from the reflection surface, the velocity of the reflected cells, and the effects of possible external factors on the cells after reflection, such as gravity, air currents, or other external forces that might act on the cells to change their speed and/or direction of motion. As an example, when choosing where to position one or more separation elements, relative to one or more reflection surfaces, the minimum vertical distance, dᵥ, from the central point of impact on the reflection surface of a stream of incident differentiated and undifferentiated cells may be calculated using the Formula dᵥ=b(√C_{d}.√Cᵤ/(√C_{d}−√Cᵤ))/sin θ, where dᵥ is the vertical distance from centre plane of impact; θᵢ is the incident angle, C_{d} and Cᵤ are the coefficient of restitution of differentiated and undifferentiated cells, respectively, and b is the beam width. Thus, for a beam width of 50 μm, incident angle of 60 degrees, Cd and Cu of 0.8 and 0.7 respectively, dᵥ is approximately 1 mm and therefore the separation between the reflection surface and the separation element may be at least 1 mm. In practice, however, the separation may be greater than this minimum difference, e.g. double the minimum distance or otherwise.

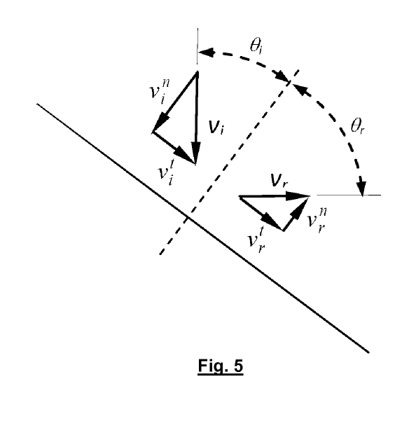

Although only one reflection surface 140 is shown in FIG. 1, a plurality of reflection surfaces may be provided. The at least two types of cells may be configured to impact and reflect from the plurality of reflection surfaces sequentially in order to increase the difference between the reflection angles of the at least two types of cells after reflecting from each surface. By increasing the difference in reflection angles, separation and collection of the at least two types of cells may be more straightforward. An example apparatus in which two reflection surfaces are provided is shown in FIG. 4, discussed further below.

As indicated, it is recognised that one or more cells of the first type of cells may still be interspersed with one or more cells of the second type of cells in cell samples, even after a first separation. In these circumstances, the separated cell samples may be considered to not be completely ‘pure’. To increase purity, one or more separated cell samples may be recirculated through the apparatus by being fed back through the nozzle 130 and subjected to the same sorting process again, or by being fed through the nozzle 130 and subjected to the sorting process with one or more variables changed, such as, but not limited to, the angle of the reflection surface relative to the incident angle, or the nozzle speed, etc. The recirculation process may be a closed loop process, where the same collection of cells is recirculated only, or an open-loop process, where additional cells to be sorted are introduced into the system at the same time as cells are being recirculated through the sorting process.

Additionally or alternatively, one or more of the separated cell samples may be subject to one or more additional sorting stages ‘upstream’ or ‘downstream’, (i.e. before or after, respectively, the sorting as described above with respect to examples herein), which additional sorting stages may employ one or more additional reflection surfaces and/or separation elements and may operate under the same sorting principles already discussed, or may employ different sorting elements, apparatus and/or sorting principles, etc. For example, a separated cell sample may be subjected to cell cytometry sorting apparatus ‘downstream’. As another example, a rough cell sorting for viable cells may be carried out ‘upstream’, which cells are then fed into apparatus as described herein.

The application of the cells to a recirculation process or to further sorting stages may be exercised automatically in certain embodiments. For example, detection apparatus may be provided to automatically detect the purity of separate cell samples, determine whether the purity meets a desired level of purity, e.g. the total number of cells includes 70% or more, 80% or more, 90% or more, or 95% or more, of one cell type only, and, on this basis, determine whether or not to subject the cells to further sorting processes. Detection of the purity of cell samples may be performed taglessly, by visual imaging and identification of cells by morphology. Alternatively, detection may be performed with tags, for example, by cell-type specific molecular tags of a fluorescent, and/or magnetic type.

It should be understood that the techniques described herein may be automated using a variety of technologies. For example, one or more of the steps described herein may be initiated, or cell sorting parameters may be adjusted, using a series of computer executable instructions residing on a suitable computer readable medium. For example, computer executable instructions may control one or more switching elements that may optionally be included in the apparatus, such as a switching element configured to turn the delivery of cells from the nozzle ‘on’ or ‘off’. As another example, computer executable instructions may control one or more motorized elements, e.g. one or more linear actuators or piezo-electric motors, that may optionally be included in the apparatus, to relatively pivot walls of a wedge element or relatively pivot the nozzle and the reflection surface to allow the incident angle to be varied, etc. Suitable computer readable media may include volatile (e.g. RAM) and/or non-volatile (e.g. ROM, disk) memory, carrier waves and transmission media (e.g. copper wire, coaxial cable, fibre optic media). Exemplary carrier waves may take the form of electrical, electromagnetic or optical signals conveying digital data streams along a local network or a publically accessible network such as the Internet

Additionally or alternatively, to increase purity, the one or more separation elements, e.g. the wedge, may be positioned or modified in a biased manner, so that they separate, at least on one side, cells that have a significantly different reflection angle to certain other reflected cells. By having a significantly different reflection angle to certain other reflected cells, those cells may be more likely to have a significantly different stiffness to the other reflected cells, making it more likely that they are of a particular cell type, for example. This approach may ensure that at least a cell sample separated to one side of the separation element has a desired level of purity, although this may be at the expense of the yield (i.e. total number of cells) of that separated sample, and the purity of one or more other separated cell samples.

In general, the desired level of purity of separated cell samples may depend on the application or end use of the separated cell samples (e.g., therapeutic use may require a greater purity than research use).

Although only two types of cells having different mechanical stiffness are shown in the example illustrated in FIG. 1, it is considered that more than two types of cells having different mechanical stiffness may be delivered for sorting. In one approach, where there are initially more than two different types of cells to be sorted, multiple sorting steps may be employed. For example, as a first step, the apparatus may be configured to sort one type of cells from two or more other types of cells. Subsequently, the two or more other types of cells may be recirculated through the apparatus, or subjected to further ‘downstream’ sorting stages, to partially or entirely sort these other types of cells from each other. The recirculation process or further sorting stages may be substantially as described above, for example.

In another approach, more than two collection areas may be provided to collect a respective one of the more than two different types of cells, after reflection from the one or more reflection surfaces. In these circumstances or otherwise, a plurality of separation elements may be used to maintain separation of the cells. For example, if there are three types of cells to be sorted, at least two separation elements may be provided to maintain three types of separated cells apart after reflection from the one or more reflection surfaces, at least one of the separation elements being located directly between two of the three cell types and at least one other of the separation elements being located between a different two of the three cell types.

In some circumstances, the different cell types present in a sample may be known prior to sorting, e.g., through fluorescent detection or visual imaging for cell morphology, and sorting may be performed to separate one cell type from one or more other cell types, and/or each cell type from the other. In other circumstances, the different cell types present in the sample may be completely or partially unknown prior to sorting and the apparatus may be used to separate the different types present as well as to optionally identify different types of cells through the sorting process, based on where the cells are collected, for example. Thus, in some circumstances, it may be assumed that, due to a particular cell being collected in a particular collection area, that cell may have particular mechanical stiffness properties, which particular stiffness properties may be indicative of its cell type.

In the example illustrated in FIG. 1, the reflection surface 140 is optionally a substantially flat, rigid and smooth surface. The reflection surface 140 may be substantially flat and smooth to the extent that the reflection surface is substantially uniform across an area of the surface where the cells will impact the surface. Therefore, a substantially uniform reflection angle of the cell can be expected, even if contact points of different cells with the surface differ slightly in position from each other, for example. However, the reflection surface could be a surface that is curved, irregular, flexible, rough or otherwise. In general, it is considered that any object against which the cells may collide with, and reflect from, may be used. When the reflection surface is not flat, the incident angle of the cells may be considered as the angle of the tangent to the surface at a contact point of the cells with the surface.

As indicated, the cells 110, 120 reflect at different reflection angles dependent on differences in mechanical stiffness. Mechanical stiffness is indicative of the resistance offered by a body to deformation. The stiffer a cell, the less it will deform upon collision with a surface, e.g., in accordance with principles of a simple mechanical spring. Since collision and deformation are, in practice, somewhat inelastic, a greater degree of deformation can correspond to a greater kinetic energy (KE) loss by a cell on collision. As a result of the differences in deformation and kinetic energy loss, cells can behave differently when they reflect from a surface. Cells with greater relative stiffness can have a greater velocity when they reflect from a surface (i.e., a greater reflection velocity) than cells with lower relative stiffness. When the cells collide with a surface at an oblique angle relative to their flow path, cells with greater relative stiffness can reflect from the surface at a smaller reflection angle to the normal of the surface (e.g., an angle closer to the incident angle relative to the normal of the surface) than cells with lower relative stiffness. By travelling at different reflection velocities and/or at different reflection angles, cells with different mechanical stiffness can travel to different positions in free space, as indicated in FIG. 1 for example, allowing physical separation of different types of cells to be achieved.

The apparatus 100 may be used to sort a variety of different types of cells, including human, mammalian or animal cells. It is not intended that apparatus or method disclosed herein be necessarily limited to sorting any particular cell type.

For example, the apparatus or method may be used to sort stem cells. Stem cells include cells that have the capacity to self renew (i.e., go through cycles of cell division while maintaining the undifferentiated state) and to differentiate into specialized cell types. Stem cells can be either totipotent or pluripotent, although multipotent, oligopotent, or unipotent progenitor cells may also be considered as stem cells. Differentiation of stem cells may increase mechanical stiffness of cells. For example, undifferentiated embryonic stem cells exhibit greater deformability (up to six times greater, for example) than fully differentiated stem cells. Cell stiffness properties of differentiated stem cells are discussed in Pajerowski, J. D., et al., Physical plasticity of the nucleus in stem cell differentiation, PNAS (2007) Oct. 2, vol. 104, no. 40, 15619-15624, for example.

Thus, the apparatus or method may be used to sort cells that have different degrees of differentiation. For example, non-differentiated stem cells may be sorted from differentiated stem cells. As another example, differentiated stem cells may be sorted from progressively more differentiated stem cells. The apparatus may also be used to sort non-differentiated or partially differentiated stem cells from terminally differentiated cells.

As an example, undifferentiated embryonic stem cells may be sorted from further differentiated counterparts. As another example, adult stem cells with stiffness characteristics varying depending on their stem cell type and degree of differentiation may be sorted.

In addition to stem cells, the apparatus or method may be used to sort other types of cells which vary in stiffness. For example, cancerous cells may have a different stiffness to their normal cell counterparts and therefore the apparatus and method may be used to separate such cells. Metastatic cancer cells, for example, are less stiff than regular mesothelial cells. By permitting sorting of such cells, the apparatus or method described herein may be used in a diagnostic technique.

In general, a cell type that is sorted from another cell type may itself include different kinds of cells. The different kinds of cells may have similar mechanical stiffness properties in comparison to the type of cells that they are to be sorted from. As one non-limiting example of this, one cell type might include a collection of different stem cells having different degrees of differentiation, and these stems cells may be sorted collectively from a cell type including terminally differentiated cells only. In general, a ‘cell type’ may include a broad range of cells that may have mechanical stiffness properties falling within in a predetermined range, for example.

Example mathematical analysis of behaviour of cells as they reflect from a surface follows. The analysis may be used to at least partially determine, for example, appropriate settings and configurations for the sorting apparatus or method, such as the incident velocity, incident angle and/or positioning and orientation of a reflection surface, one or more separation elements and/or collection devices for collecting cells, or otherwise. However, it is conceived that various approaches may be taken to analysing and predicting behaviour of cells, which may use different mathematical formulae to the formulae presented and/or use experimentation and observation, for example. Overall, understanding mathematical theory behind the behaviour of cells is not necessary to implement embodiments and examples disclosed herein.

A value for the work done Win deforming a cell upon impact with a surface can be determined using Formula 1:

W=−πE{p³[A−1/3 ln(p)]+p[r² ln(p)−B]+C} Formula 1

where p=(r−d), A=1/3 ln(r)+1/9, B=r² ln(r)+r² and C=8/9r³, and where E is the modulus of elasticity of the cell, r is the radius of the cell and d is the total deformation of the cell.

Formula 1 assumes at least that cells are approximately spherical, the stiffness properties of the cells are homogeneous throughout each cell, the collision surface is flat, and there is no cell slippage upon collision with the surface.

Formula 1 may be used to determine an approximate value for the total deformation of a cell by substituting quantitative values for E, r, m and v using the relation:

W=KE=1/2mv² Formula 2

where m is the cell mass and v is the velocity of the cell incident on the surface (incident velocity).

Formula 1 indicates that, at least when velocities and masses of cells are substantially the same, and thus their kinetic energies are also substantially the same, cells of higher relative stiffness (with higher Elastic modulus) will be deformed less than cells of lower relative stiffness (with lower Elastic modulus).

Assuming that, in practice, cell deformation is not energy conserving due to losses in the collision with the surface, larger relative deformation will result in larger relative kinetic energy loss for cells of lower relative stiffness, resulting in a relatively lower reflection velocity for such cells. With reference to FIG. 5, when deformation is normal to the impact surface, only the normal component, vᵣⁿ, of the reflection velocity, vᵣ, may be affected by the collision, and components of the incident and reflection velocities tangential to the surface, vᵢ^{t} and vᵣ^{t}, may not be substantially affected. When vᵣⁿ is less than the corresponding normal component of the incident velocity, vᵢⁿ, and when vᵢ^{t} and vᵣ^{t} are substantially identical, the angle of reflection of the cells, θᵣ, will not equal the angle of incidence θᵢ. Specifically, a relatively greater reduction in vᵣⁿ, as a result of relatively greater deformation and energy loss, will cause the cells of relatively lower stiffness cells to reflect from the surface at a reflection angle relative to the normal that is greater than the reflection angle of the cells of relatively higher stiffness.

As indicated, a value for the deformation, d, for different cell types can be calculated using Formula 1. To calculate a reflection angle, θᵣ, for cells, d can be inserted into Formula 3, which assumes that the loss of energy in the collision is proportional to the effective strain (effective strain being expressed as the ratio of the deformation over the cell diameter).

KE-reflected=[1−(d/cell diameter)]×KE-incident Formula 3

In Formula 3, KE-reflected and KE-incident describe the kinetic energy of the cells normal to the surface, after and before reflection. Referring to Formula 2, values for KE-incident can be determined from the cell mass and incident velocity (which may correspond substantially to the nozzle velocity of a delivery device), taking into consideration the incident angle of the cell on the reflection surface. By obtaining KE-reflected, again with reference to Formula 2, the reflection velocity of the cell normal to the surface, vᵣⁿ, can be calculated and reflection angles can then be calculated using the following formula, Formula 4:

θᵣ=tan⁻¹(vᵣ^{t}/vᵣⁿ) Formula 4

Taking non-differentiated and differentiated stem cells as a non-limiting example of cells that may be sorted, and using the following possible properties for stem cells, and example approximate values for incident velocities and incident angle, deformation, d, velocities, vᵣⁿ, and reflection angles θᵣ, can be calculated as follows:
Mass: m=0.63×10⁻¹² kg (cell mass)
Radius: r=5×10⁻⁶ m (cell diameter 10 μm)
Elastic modulus: E_{n-dif}=900 Pa (undifferentiated cell)
   E_{dif}=5400 Pa (differentiated cell)
Incident velocity: vᵢ=0.75 m/s; vᵢⁿ=0.53 m/s; vᵢ^{t}=0.53 m/s
Incident angle: θᵢ=45°
Deformation d_{n-dif}=3.5 μm (undifferentiated cell)
   d_{diff}=1.9 μm (differentiated cell)
Reflection velocities v_{r n-dif}ⁿ=0.426 m/s
   v_{r dif}ⁿ=0.475 m/s
Reflection angles θ_{n-dif}=51.2°
   θ_{r dif}=48.2°

The calculations indicate a difference in reflection angle for the differentiated and non-differentiated cells of about 3 degrees based on this example mathematical approach.

Nonetheless, in practice, cells are not perfectly elastic and they more closely match a viscoelastic model. An alternative measure of energy loss during impact may therefore be coefficient of restitution, which may range from about 0.5 for cells of higher relative stiffness to 0.1 for the cells of lower relative stiffness, for example. Taking this into account, a larger difference in reflection angle between different types of cells may be predicted. The difference may be 20 degrees, rather than 3 degrees, for example.

Furthermore, in practice, a population of cells may not be completely homogenous in terms of such physical properties as size, density and shape, and some variation is expected. This can affect the reflection velocities and angles.

Referring to Tables 1a and 1b, and in consideration of stem cell and apparatus parameters as discussed herein previously, a variation of 10% in cell radius can vary the difference in the reflection angles of undifferentiated and differentiated stem cells by about 3%, for example. Similarly, a variation of 20% in density can vary the difference in the reflection angles of undifferentiated and differentiated stem cells by about 10%, for example.

TABLE 1a

| Radius | Radius | density | angle | |
|---|---|---|---|---|
| (micron) | variation | (kg/m\hat{ }3) | difference | % change |
| 5 | −10% | 1200 | 3.1 | 0 |
| 5.5 | mean | 1200 | 3.1 | 0 |
| 6 | 10% | 1200 | 3 | −3 |

TABLE 1b

| Radius | density | density | angle | |
|---|---|---|---|---|
| (micron) | variation | (kg/m\hat{ }3) | difference | % change |
| 5 | −20% | 1000 | 2.8 | −10 |
| 5 | mean | 1200 | 3.1 | 0 |
| 5 | 25% | 1500 | 3.4 | 10 |

To address variability in cell population, one or more separated cell samples may be cycled through the same apparatus more than once, or may be subjected to one or more additional sorting stages ‘downstream’, which additional sorting stages may employ one or more additional reflection surfaces and separation elements and operate under the same sorting principles or otherwise. In general, subjecting cells to additional sorting processes may increase enrichment or sample purity and increase cell yields. As an alternative, or additionally, incident velocity or incident angle may be varied to tune the apparatus for particular populations of cell types.

As indicated, in addition to physical properties of cells to be sorted, the reflection velocities and reflection angles can be affected by the incident velocity and incident angle of the cells, as they collide with the reflection surface. The incident velocity may largely depend on the velocity of the cells as they leave the nozzle, i.e., the nozzle velocity. The incident velocity may be kept below a level at which little or no damage to cells will occur upon collision with the reflection surface, or with any elements of the apparatus further downstream. For example, the incident velocity of the cells may be of the same order of magnitude as used in existing FACS equipment as discussed above, to the extent that it does not damage cells. Nonetheless, cell damage may also be reduced or prevented by encasing the delivered cells in fluid droplets and this may permit greater incident velocities to be used. The droplets can include cells with sheath fluid such as water encasing them. The droplets may be located in air as they move towards and away from the reflection surface. The combination in a droplet of the fluid and a cell can have a different stiffness to the cell by itself. However, the relative stiffness of droplets containing cells of different stiffness, within the same type of fluid, can remain approximately the same. Although, using conventional FACS nozzle arrangements, cells will normally be encased in a sheath fluid at least during delivery to the one or more reflection surfaces, it is recognized that a flow of cells may produced by alternative means. It is considered that delivery of a flow of cells, which cells are not encased in fluid droplets, may utilized in the apparatus and methods described herein. The cells may be delivered in a gas medium only, such as air, for example. Alternatively, rather than being encased in respective fluid droplets, the cells may disposed in a larger fluid medium, as they travel to and/or from the reflection surface. As an example, the cells may be delivered to the reflection surface, and to the collection areas, via in one or more microfluidics channels containing fluid.

The incident velocity may be of an order of magnitude lower than stresses experienced by cells in various natural processes (e.g., cell division). The incident angle may be chosen to ensure a maximum difference in the reflection angle, dependent on the types of cells being sorted, for example. It is considered that an incident angle of between about 30 and 45 degrees, or 40 degrees and 50 degrees, e.g., 45 degrees, may be appropriate, but other angles may be used. Although the incident angle is fixed in FIG. 1, the apparatus may be configured such that the angle is variable. The reflection surface and/or the nozzle may be relatively pivotable, for example, to allow the incident angle to be varied. To achieve this, the nozzle may be pivotable relative to a support surface on which the apparatus is located and/or the reflection surface may be pivotable relative to the support surface. In general, to increase sorting efficiency, optimization may be performed to determine appropriate incident velocity, incident angle, reflection surface materials and collection area positioning, etc., depending on the types of cells being sorted, for example.

FIG. 2 illustrates another example apparatus 200 for sorting cells. The apparatus 200 is configured similarly to the apparatus of FIG. 1, and employs a similar method to sorting cells as the apparatus of FIG. 1. However, the incident and reflection flow paths 151, 152, 153 are shown as enclosed within a housing 210 in this example. The housing 210 in this example, or a housing in any other example, may function to at least partially shield the cells, as the cells are delivered to the one or more reflection surfaces and/or after they reflect from the one or more reflection surfaces, from external factors that could affect the apparatus, e.g., change the speed of the cells or change the reflection angle in an unpredictable manner, and/or damage the cells, etc. External factors may include air currents, excessive heat or cold etc., or dirt or other contaminants. The housing may entirely or partially surround the flow paths of the cells before and/or after reflection from the one or more reflection surfaces. The housing may include walls, panels or other elements capable of at least partially shielding the cells. In some embodiments, the housing 210 has a plurality of walls that optionally define one or more conduits through which the cells 110, 120 can travel. A conduit may be any element defining a passage that the cells can travel through or along, and may be providing by ducting, tubing, channels or other devices. In some embodiments, one or more of the walls of the housing 210 also optionally define the one or more reflection surfaces 140. The one or more conduits as shown in FIG. 2 include a delivery conduit 220, which provides a passage through which the cells can be delivered from the nozzle 130 to the reflection surface 140. In this example, the delivery conduit 220 optionally extends vertically, with the nozzle 130 located at a top portion of the delivery conduit 220 so as to deliver cells along a substantially vertical, downward, flow path. The bottom portion of the delivery conduit 220 optionally terminates at or adjacent to the reflection surface 140. The housing 210 further includes at least two reflection conduits 231, 232. The at least two reflection conduits 231, 232 provide passages through which the at least two different types of cells 110, 120 can travel after being reflected. In this example, the two reflection conduits 231, 232 optionally extend substantially laterally from the reflection surface 140 along respective paths that, at least adjacent the reflection surface 140, follow the diverging reflection flow paths 152, 153 of the two different types of cells 110, 120 after being reflected. In combination, adjacent walls of the two reflection conduits 231, 232 provide a wedge 240 with a similar shape and function to the wedge 160 described with respect to FIG. 1. The reflection conduits 231, 232 can be connected to respective containers for holding the sorted cells or to further sorting stages, for example.

One or more of the conduits may have a cross-section perpendicular to their elongation direction that is square, rectangular, circular or otherwise. The internal walls of the conduits may be smooth and/or provided without sharp corners (e.g. acute angles) to minimize cells or droplets containing cells from getting stuck, attached, and/or damaged. To provide smooth corners, the corners may be rounded or filleted. Where rounded or filleted corners are provided, the radii of these corners may be greater than that of the cells or droplets to reduce chances of sticking The shapes and/or materials of the conduits may be chosen so that the conduits do not damage cells passing therethrough by either chemical reaction or by physical damage.

The size of the conduits may depend on the size or number of nozzles and/or the nature of the cells being sorted, for example. If a single 20 μm nozzle is used, the delivery conduit 220 may have a 40 μm×40 μm square cross-section and the reflection conduits may have a 30 μm×40 μm rectangular cross-section, for example. Multiple nozzles may be used in apparatus disclosed herein to increase the number of cells being sorted in a given time. The multiple nozzles may be provided side-by-side and may deliver cells through the same conduit(s) such that they are incident on the same reflection surfaces and collected in the same collection areas, etc., or the nozzles may deliver the cells through different respective conduits, such that they are incident on different respective reflection surfaces and collected in different collection containers, etc. When different conduits, etc., are provided, in essence, a plurality of apparatuses as illustrated in the Figures or otherwise may be used in parallel, to increase the number of cells sorted in a given time.

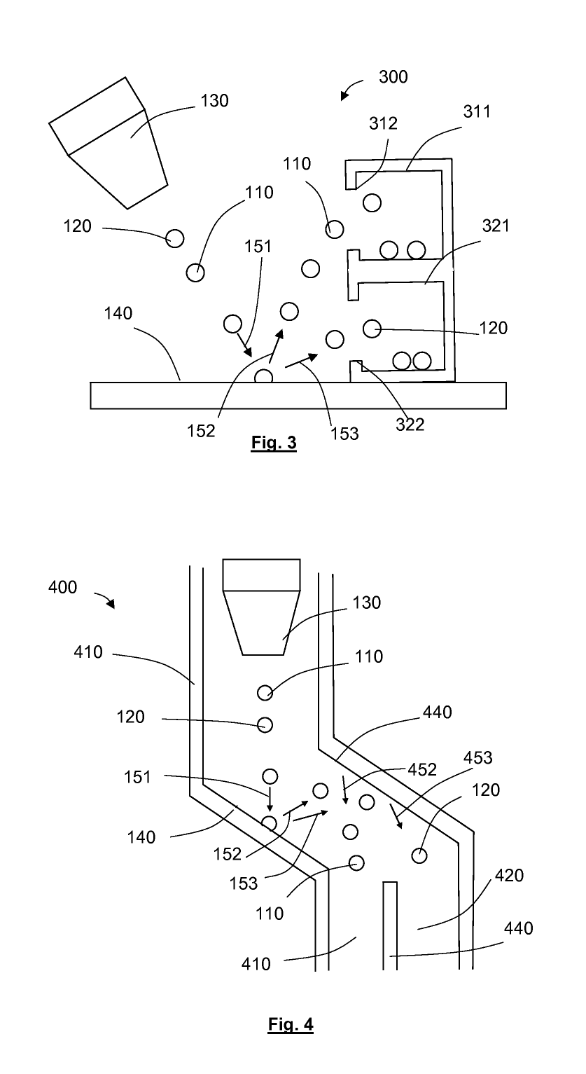

FIG. 3 illustrates a further example apparatus 300 for sorting cells. The apparatus 300 employs similar principles for sorting cells as the apparatus 100, 200 of FIGS. 1 and 2. However, in this example the reflection surface 140 is shown disposed substantially horizontally, and the nozzle 130 is shown configured to deliver cells along an incident flow path 151 that is at an oblique angle relative to the horizontal. In general, while the reflection surface 140 is at an oblique angle relative to the incident flow path, a variety of different orientations of the incident flow path 151 and the reflection surface 140, relative to the coordinates of free space, are conceivable. For example, in addition to the incident flow path being substantially vertical as shown in FIG. 1, or at an angle relative to the horizontal (and vertical) as shown in FIG. 2, it may also be substantially horizontal. Nonetheless, to maximise incident velocity, and to ensure a reasonably focussed stream of cells incident on the reflection surface, a substantially vertical incident flow path, e.g. as shown in FIG. 1, may be preferred in some embodiments.

In this example shown in FIG. 3, the apparatus optionally does not employ a wedge or conduits to collect the reflected cells, but includes at least two collection containers 311, 321, each having a respective opening 312, 322 that aligns with the reflection flow paths 152, 153 of the respective types of cells 110, 120. The openings 312, 322 may be configured so that cells cannot spill out from the containers 311, 321. The collection containers 311, 321 can be provided by any device that the cells travelling along the respective reflection flow paths 152, 153 can enter into, or land upon, and which can hold or store the separated cell samples apart from each other. In this example, the collection containers 311, 321 are provided by a box-shaped device with a central dividing wall. However, in alternative examples, the two collection containers 311, 321 may be provided by separate boxed-shaped devices. In this or other examples, chambers, cups, dishes, plates, cones or other devices may alternatively or additionally be used as collection containers.

FIG. 4 illustrates a further example apparatus 400 for sorting cells. The apparatus 400 employs similar principles for sorting cells as the apparatus 100, 200 and 300 of FIGS. 1, 2 and 3. However, in this example, in addition to a first reflection surface 140, at least a second reflection surface 440 is provided. The first and second reflection surfaces 140, 440 are provided by opposing inside surfaces of a housing 410, which surfaces both extend at an oblique angle relative to the flow path 151 of the cells delivered from the nozzle 130. The arrangement is such that, after delivery from the nozzle 130, the different types of cells 110, 120 are first incident on the first reflection surface 140 and, after reflecting from the first reflection surface 140, the different types of reflected cells 110, 120 travel along different respective flow paths 152, 153, and are incident on the second reflection surface 140 at different positions. Subsequently, after reflecting from the second reflection surface 440, the different types of reflected cells 110, 120 travel along further different respective flow paths 452, 453 towards respective collection areas 410, 420 defined between outer walls of the housing 410 and an inner central wall that acts as a separation element 440. The difference in angle between the flow paths 452, 453 of the different types of cells 110, 120 immediately after reflecting from the second reflection surface 440 is greater than the difference in angle between the flow paths 152, 153 of the different types of cells 110, 120 immediately after reflecting from the first reflection surface 140. Accordingly, the provision of the at least two reflection surfaces 140, 440 serves to amplify the difference in the angle between the flow paths of the different types of cells 110, 120. This can ensure greater separation between the different types of cells 110, 120 after reflection, making collection of the different types of cells at respective collection areas more straightforward.

In the examples provided, sorting of cells is dependent on the stiffness of different types of cells and therefore damage to cells may be significantly reduced or avoided in comparison to approaches in which a marker is applied to cells. Furthermore, the purity of sorted or separated cell samples may reach 70% or more, 80% or more, 90% or more or 95% or more. This may be achieved with a difference in reflection angle for different cell types of at least 2 degrees, 3 degrees, 5 degrees, or 10 degrees, for example. Purity may be increased by tuning of the apparatus or introducing multiple processing stages in the apparatus as discussed. The rate of cells being sorted may be higher in comparison to approaches where markers are applied to cells, since a marking stage may be eliminated and multiple nozzles delivering cells may be used.

It will be appreciated that numerous variations and/or modifications may be made to the examples. For instance, a variety of different configurations of elements of the apparatus described in the examples and as illustrated in the Figures, including the nozzles, housings, separation elements, reflection surfaces or collection containers, etc. is conceivable. Furthermore, such elements of the apparatus, described with respect to one example, may be interchangeable with one or more corresponding elements of one or more other examples, or may be provided as additional elements of one or more other examples. For example, collection containers similar or identical to the collection containers of the apparatus illustrated in FIG. 3 may be employed in the apparatus illustrated in FIG. 1, or a housing similar or identical to the housing of the apparatus illustrated in FIG. 2 may be employed in the apparatus illustrated in FIG. 3, for example. The examples are, therefore, to be considered in all respects as illustrative and not restrictive of subject matter disclosed.

1. Apparatus for sorting cells, comprising:
a cell delivery device configured to deliver two or more cell types along a flow path, wherein each cell type of the two or more cell types comprises a different mechanical stiffness and
a base having a flat reflection surface that lies in a single reflection surface plane at an impact angle, wherein the reflection surface is configured to contact the flow path and differentially reflect the two or more cell types based on the mechanical stiffness; and
two or more collection area positioned to receive the two or more cell types, wherein each cell type of the two or more cell types is separately collected based on the angle of reflection from the reflection surface

2. The apparatus of claim 1, wherein the two or more cell types are stem cells.

3. The apparatus of claim 2, wherein the stem cells comprise undifferentiated stem cells differentiated stem cells.

4. (canceled)

5. The apparatus of claim 1, wherein the two or more cell types are delivered in fluid droplets.

6. The apparatus of claim 1, wherein the two or more collection areas further comprise a first collection area conduit and a second collection area conduit.

7. The apparatus of claim 6, wherein the first collection area conduit and the second collection area conduit are separated by a wedge.

8. The apparatus of claim 7, wherein the wedge apex angle is configured to substantially separate different cell types based on the angle of reflection from the reflection surface.

9. The apparatus of claim 7, wherein the wedge apex angle is from 1 to 20 degrees.

10. The apparatus of claim 7, wherein the wedge apex angle is about 3 degree.

11. (canceled)

12. The apparatus of claim 1, wherein the reflection surface is a substantially solid surface.

13. The apparatus of claim 1, wherein the impact angle of the reflection surface is from 40 to 50 degrees relative to the flow path.

14. The apparatus of claim 1, wherein the reflection surface is movable relative to the flow path.

15. The apparatus of claim 1, wherein the two or more collection areas are moveable.

16. A method of sorting cells, comprising:
directing a flow path comprised of two or more cell types to contact a flat reflection surface that lies in a single reflection surface plane at an impact angle, wherein each cell type of the two or more cell types comprises a different mechanical stiffness, and wherein the mechanical stiffness is proportional to a reflection angle;
separating the two or more cell types based on the mechanical stiffness and the reflection angle; and
collecting the two or more cell types.

17-20. (canceled)

21. The method of claim 16, wherein the separating further comprises a separating element having an apex and an apex angle.

22. The method of claim 21, wherein apex angle is from 1 to 20 degrees.

23. The method of claim 21, wherein apex angle is about 3 degrees.

24. The method of claim 16, wherein the reflection surface is a substantially solid surface.

25. The method of claim 16, wherein the impact angle of the reflection surface is from 40 to 50 degrees relative to the flow path.

26. The method of claim 16, wherein the cells are stem cells.
